(19) United States
(12) Patent Application Publication
(10) Pub. No.: US 2017/0087377 A1
(43) Pub. Date: Mar. 30, 2017

(54) Transnasal Delivery of Low Level Light Via the Sphenoidal Sinus to Irradiate the Substantia Nigra

(71) Applicant: DEPUY SYNTHES PRODUCTS, LLC, Raynham, MA (US)

(72) Inventors: Martin Pfleiderer, Auvernier (CH); Yanik S. Tardy, Geneveys-sur-Coffrane (CH); Blaise Lovisa, Orsìeres (CH)

(21) Appl. No.: 14/867,646

(22) Filed: Sep. 28, 2015

(51) Int. Cl. A61N 5/06

(52) U.S. Cl. CPC A61N 5/0603; A61N 5/0622; A61N 2005/0607; A61N 2005/063; A61N 2005/0662; A61N 2005/0659

(57) ABSTRACT

A transnasal delivery method for treating or preventing Parkinson's disease using photobiomodulation. An optical system is provided including a light source and an optical fiber. The optical system is advanced through the nasal cavity until a distal end of the optical fiber is positioned inside the sphenoidal sinus. Then the light source is activated to irradiate substantia nigra brain tissue with an effective amount of light in the treatment or prevention of Parkinson's disease.

### BACKGROUND OF THE INVENTION

Field of the Invention

The present invention relates to a system and device for transnasal delivery of low level light. In particular, the present invention is directed to a system and device for transnasal delivery via the sphenoidal sinus of low level light to the substantia nigra in the treatment of Parkinson's disease.

Description of Related Art

Low level light therapy, also known as phototherapy or photobiomodulation, has been a recognized treatment of many diseases and disorders of the human body including those of the brain. Heretofore, photobiomodulation of the substantia nigra, an anatomical structure inside the brain stem, for the treatment of Parkinson's required a suitable access path through the brain to position the optical light source at a desired location in the brain proximate the target tissue of the substantia nigra. Such previously recognized access paths were directed through the frontal lobe of the brain thus requiring brain surgery.

The intranasal delivery of red light for the treatment of Alzheimer's disease by illuminating diseased brain cells is disclosed in U.S. Pat. No. 7,351,253, assigned to Codman & Shurtleff and herein incorporated by reference in its entirety. Positioning the distal end of the optical fiber within the nasal cavity allows for illumination of red light to the olfactory bulb via the substantially permeable cribriform plate portion of the nasal cavity in the treatment of brain cells affected by Alzheimer's disease. In accordance with the patented procedure used in the treatment of Alzheimer's disease, the distal end of the optical fiber at all times remains only within the nasal cavity. Accordingly, only that amount of irradiated light able to permeate through the cribriform plate portion of the nasal cavity is able to enter the brain. As expressly recognized in the patented invention, the irradiated red light generated by the optical fiber is only able to penetrate the gray matter to a depth of up to a centimeter. Accordingly, using the patented device a sufficient amount of irradiated light would not be able to reach deep into the brain to illuminate substantia nigra required in the treatment of Parkinson's disease. It is therefore desirable to develop a photobiomodulation system and method that does not require brain surgery through the frontal lobe but, nevertheless, transnasally irradiates via the sphenoidal sinus the substantia nigra whereby a sufficient amount of irradiated light is able to penetrate deeper in the brain as required for treatment of Parkinson's disease.

### SUMMARY OF THE INVENTION

An aspect of the present invention is directed to a system and method for photobiomodulation of the substantia nigra via intranasal delivery (i.e., delivery via the sphenoid sinus) in the treatment of Parkinson's disease without the need for surgical intervention into the brain (i.e., no need to access the brain via the front lobe or any other surgical intervention into the brain). The only surgery that typically may be required is Ear, Nose and Throat (ENT) surgery for enlarging/dilating/widening of the natural paranasal ostium sinus used in conventional sinusitis treatment procedures performed under general or local anesthesia. During access surgery, due to anatomic variation from patient to patient in those patients with relatively thick bone in the area behind the sphenoidal sinus, it may be advantageous to precisely decorticate such bone (without violating or damaging the dura) in order to reduce light absorption by the bone.

Another aspect of the present invention is directed to a system and method for photobiomodulation of the substantia nigra via the sphenoid sinus in the treatment of Parkinson's disease that eliminates the need for an implanted device.

Still another aspect of the present invention is directed to a system and method for photobiomodulation of the substantia nigra via the sphenoid sinus in the treatment of Parkinson's disease that occurs with repeated treatments performed in an outpatient setting, and may be performed under local anesthesia at relative low cost (without the need for an inpatient setting under general anesthesia).

In particular, the present claimed invention is directed to a transnasal delivery method for treating or preventing Parkinson's disease using photobiomodulation. An optical system is provided that includes a light source and an optical fiber. The optical system is advanced through the nasal cavity until a distal end of the optical fiber is positioned within the nasal cavity. Then the light source is activated to irradiate substantia nigra brain tissue with an effective amount of light in the treatment or prevention of Parkinson's disease.

While another aspect of the present claimed invention is directed to a transnasal delivery method for treating or preventing Parkinson's disease using photobiomodulation. An optical system is provided that includes a light source. The optical system is advanced through the nasal cavity until a distal end of the light source is positioned within the nasal cavity. Then the light source is activated to irradiate substantia nigra brain tissue with an effective amount of light in the treatment or prevention of Parkinson's disease.

### BRIEF DESCRIPTION OF THE DRAWING

The foregoing and other features of the present invention will be more readily apparent from the following detailed description and drawings of illustrative of the invention wherein like reference numbers refer to similar elements throughout the several views and in which:

FIG. 1 is a graphical representation of measured values of fluence rate along an antero-superior direction at four different aspects of the substantia nigra (e.g., substantia nigra anterior right; substantia nigra posterior right; substantia nigra posterior left; substantia nigra anterior left) with low level light at a wavelength of 808 nm delivered transnasally in accordance with the present invention;

FIG. 2 is an illustration of fluence rate as a proportion to the emitted fluence rate in four different aspect of the substantia nigra (e.g., substantia nigra anterior right; substantia nigra posterior right; substantia nigra posterior left; substantia nigra anterior left) with low level light, at a wavelength of 808 nm delivered transnasally in accordance with the present invention; and

FIGS. 3A-3D is a prior art set of illustrative devices for enlarging/dilating/widening of the natural paranasal ostium sinus; and

FIG. 4 is an exemplary embodiment of the device used in accordance with the present inventive methodology for transnasal delivery of low level light radiation from inside the sphenoid sinus to irradiate the substantia nigra in the treatment of Parkinson's disease.

### DETAILED DESCRIPTION OF THE INVENTION

The present invention is directed to a system and method for transmission of low level light (preferably in the wave length range of approximately 650 nm-approximately 1000 nm, most preferably, between approximately 670 nm-approximately 810 nm) delivered transnasally via the sphenoidal sinus to the substantia nigra for photobiomodulation of brain cells in the specific treatment of Parkinson's disease. The present inventive system and method does not require brain surgery on the frontal lobe or any other portion of the brain. Furthermore, the present invention system and method for transnasal photobiomodulation in the treatment of Parkinson's disease wherein the irradiated light does not cross the cribriform plate or irradiate the olfactory bulb.

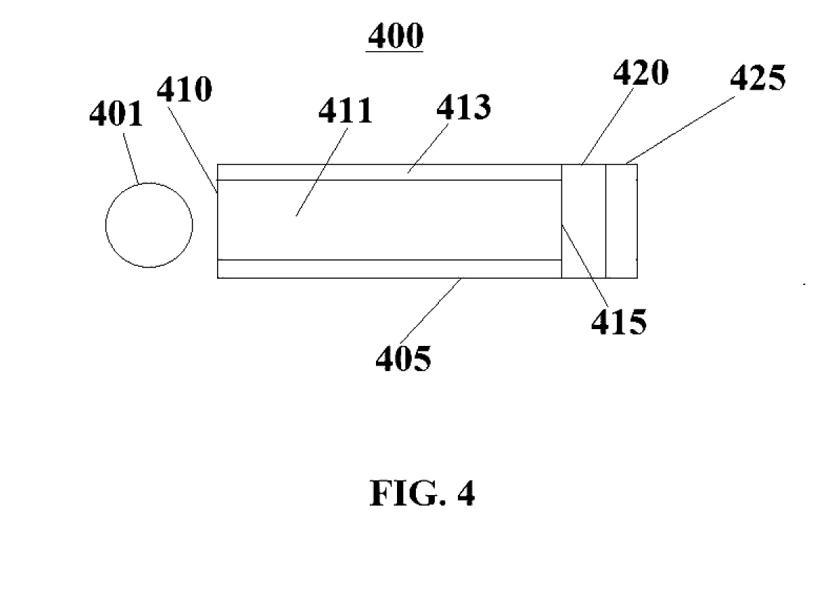

FIG. 4 is an exemplary embodiment of an optical system 400 used in accordance with the present invention during transnasal delivery of photobiomodulation to the substantia nigra from inside the sphenoid sinus for the treatment of Parkinson's disease. An optical fiber 405 receives light produced by a light source 401 (e.g., a laser light source) to illuminate the substantia nigra. Preferably, the light source 401 is selected to have a wavelength within the range of approximately 650 nm-approximately 1000 nm, most preferably in the range of approximately 670 nm-approximately 810 nm. Optical fiber 405 has a proximal end 410 and an opposite distal end 415 insertable inside the nasal cavity, preferably to a location in which the distal end of the optical fiber is entirely within the sphenoid sinus. The optical fiber comprises an inner core 411 surrounding by a cladding layer 413. Contrary to the patented device and method in U.S. Pat. No. 7,351,253 used in the treatment of Alzheimer's disease calling for the illumination of as wide an area as possible of the olfactory bulb via the cribriform plate, the treatment of Parkinson's disease in accordance with the present inventive optical system requires the irradiated light be shaped/focused/targeted in order to reach the substantia nigra disposed deep in the brain while simultaneously minimizing burning/damaging/degrading of the surrounding tissue. One or more optical components may be employed at the distal end of the optical fiber to insure that a sufficient amount of irradiated light reaches the substantia nigra with de minimis, if any, damage to the surrounding tissue. By way of illustrative example, a frontal diffuser 420 (e.g., the Frontal Light Distributor Model FD manufactured by Medlight S.A.) in series with one or more lenses 425 may be employed. The frontal diffuser maximizes the irradiated light exiting from the optical fiber to insure that a sufficient amount penetrates to the desired depth to reach the substantia nigra, while the one or more lenses homogenize the light emanating from the tip of the frontal diffuser to an optimum shape/focus/target to cover, without exceeding, the perimeter of the substantia nigra (e.g., approximately 2 cm) thereby minimizing damage to the surrounding tissue.

Initial ENT surgery is preferably employed to permanently enlarge or widen the sinus ostium thereby facilitating access of the distal end of the present inventive optical system (including the distal end of the optical fiber, the frontal diffuser and the one or more lenses) inside the sphenoid sinus. This initial procedure is typically carried out in a medical facility under general or local anesthesia using any conventional methodology for enlarging/dilating/widening the paranasal sinus ostium. One exemplary conventional methodology for enlarging/dilating/widening a natural paranasal sinus ostium that has not been previously surgically altered is disclosed in U.S. Pat. Nos. 7,462,175 & 7,500,971 (assigned to Acclarent, Inc.), each of which is herein incorporated by reference in its entirety. Other systems and methods for enlarging/dilating/widening the natural, paranasal sinus ostium that has not been previously surgically altered are contemplated and within the intended scope of the present invention,

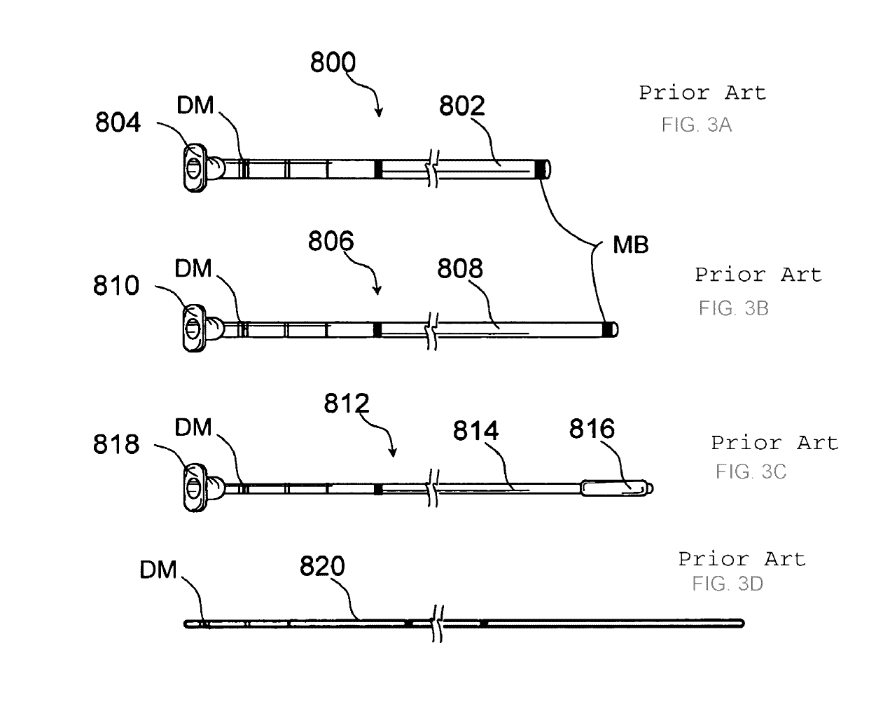

FIGS. 3A-3D show perspective views of a set of illustrative conventional devices to enlarge/dilate/widen a natural paranasal sinus ostium in accordance with the patented method of U.S. Pat. Nos. 7,462,175 & 7,500,971. As previously mentioned, other systems and methods for enlarging/dilating/widening of the natural paranasal sinus ostium are contemplated and within the intended scope of the present invention. Referring to FIGS. 3A-3D, a guide catheter 800 comprises a shaft 802 comprising a threaded luer 804 disposed at a proximal end of shaft 802. Distal end of shaft 802 preferably comprises a radio-opaque marker band MB to enable the physician to identity the tip of shaft 802 in a fluoroscopic image. The distal end of shall 802 may be substantially straight or may comprise one or more bent or angled regions. One or more distance markings DM may also be located on the shaft 802. An optional subselective catheter 806 may also be present in the set of devices. Subselective catheter 806 comprises a shaft 808 comprising a threaded luer 810 at the proximal end of shaft 808. Inner diameter of shaft 808 is smaller than inner diameter of shaft 802. Distal end of the shaft 808 comprises a radio-opaque marker band MB to enable the physician to identify the tip of shaft 808 in a fluoroscopic image. Distal end of shaft 808 may be substantially straight or may comprise one or more bent or angled regions. One or more distance markings DM may also be located on the shaft 808. Working device 812 comprises a shaft 814 comprising a working element 816 located on distal region of shaft 814 and a threaded luer 818 located on proximate end of shaft 814. In this example the working element 816 is preferably a dilating balloon. The distal end of shaft 814 may be substantially straight or may comprise a bent or angled region. One or more distance markings DM may also be located on shaft 814. The set of devices further includes a guidewire 820. Guidewire 820 may be substantially straight or may comprise a bent or angled region. One or more distance markings DM may also be located on the guidewire 820. The enlargement system may be configured, as desired, to include any one or more of the combination of instruments of the set of devices illustrated in FIGS. 3A-3D.

In one embodiment of the method of using the abovementioned set of devices, guide catheter 800 is introduced into the patient's body so that the distal end of the guide catheter 800 is in the vicinity of an anatomical opening (e.g., ostium) of an anatomical region (e.g., paranasal sinus). Thereafter, guidewire 820 is introduced through guide catheter 800 into the anatomical region (e.g., paranasal sinus). If necessary, guide catheter 800 may be removed and the smaller subselective catheter 806 may be introduced over guidewire 820 into the paranasal sinus and enlargement/widening/dilation of the ostium sinus is achieved by dilating the working device 812 (including dilating balloon 816). In another embodiment of a method of using the aforementioned set of devices, subselective catheter 806 may be introduced into a patient's body so that the distal end of the subselective catheter 806 is in the vicinity of an anatomical opening (e.g., an ostium) of an anatomical region (e.g., paranasal sinus). Then guide wire 820 is introduced through subselective catheter 806 into the anatomical region (e.g., paranasal sinus). Thereafter, subselective catheter 806 is removed. Larger guide catheter 800 is then introduced over guidewire 820. Working device 812 (including dilating balloon 816) is then introduced over guidewire 820 into the paranasal sinus and a diagnostic or therapeutic procedure is performed by working device 812. This last method embodiment enables a user to introduce a larger working device 812 in the anatomical region.

During access surgery, due to anatomic variation from patient to patient in those patients with relatively thick bone in the area behind the sphenoidal sinus, it may be advantageous to precisely decorticate such bone (without violating or damaging the dura) in order to reduce light absorption by the bone.

Once the paranasal sinus ostium has been enlarged/dilated/widened, any subsequent photobiomodulation treatment of the substantia nigra from inside the sphenoid sinus in accordance with the present invention advantageously may be performed in an outpatient setting under local anesthesia in relatively short period of time (e.g., approximately one hour). The present inventive optical system for the treatment of Parkinson's disease in accordance with the present invention may advantageously be introduced into the patient's sinus, on an as needed basis thereby eliminating the need for a permanently implanted device.

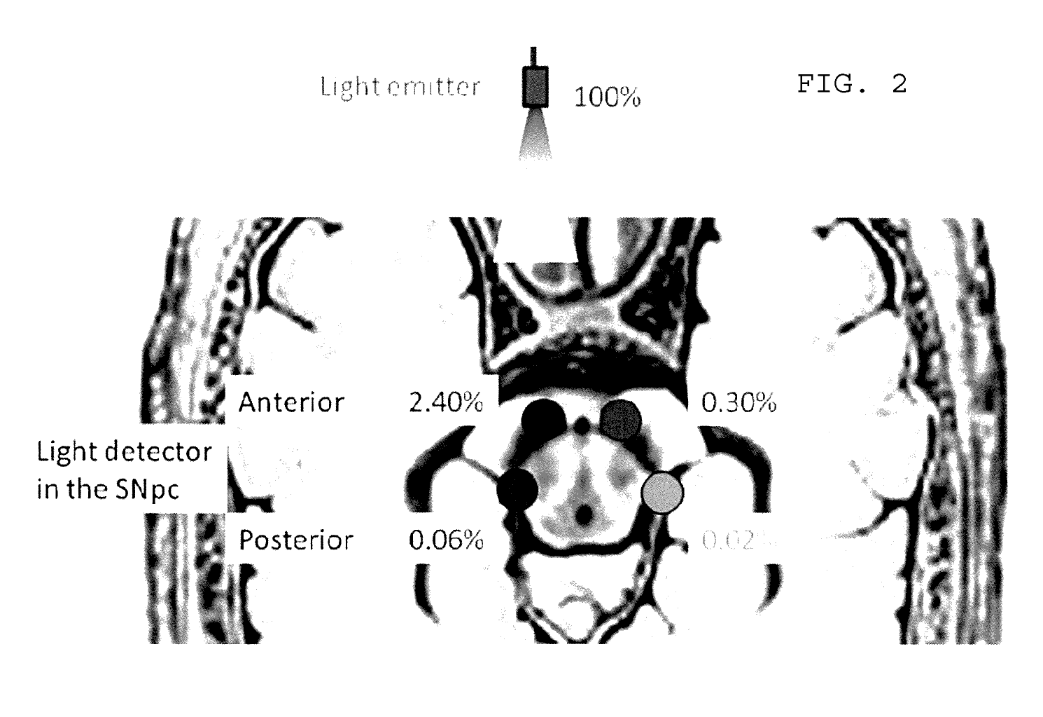

Once the optical fiber, light diffuser and one or more lenses have been introduced via the patient's sinus inside the sphenoidal sinus, the light exiting therefrom irradiates the target tissue of substantia nigra deep within the brain tissue. Specifically, during treatment, the irradiated light enters the bone and brain tissue via the sphenoidal sinus. The light that enters the bone and brain tissue is partially absorbed and scattered, whereby a portion thereof reaches the target tissue of the substantia nigra. FIG. 2 is a graphical representation of fluence rate along an antero-superior direction at four different aspects of the substantia nigra using an optical fiber whose wavelength is 808 nm. The four different aspect of the substantia nigra, as illustrated in FIG. 3 are the following: (i) substantia nigra anterior right; (ii) substantia nigra posterior right; (iii) substantia nigra posterior left; and (iv) substantia nigra anterior left.

Measurements on human species indicate a fluence rate of between approximately 0.1 mW/cm² and approximately 10 mW/cm² at different locations inside the substantia nigra. The highest fluence levels have been detected in locations that are closest to the optical light source inside the sphenoidal sinus. Based on this experimental data, approximately 0.1%-approximately 1.0% of light energy emitted by the optical fiber and light diffuser reaches the substantia nigra. This measured fluence rate in the target tissue of the substantia nigra is within the range of energy densities found to be efficient therapeutically for the treatment of Parkinson's disease using photobiomodulation. Moreover, the maximum fluence rate in the tissue closest to the optical fiber light source is within a safe range in which there is no, or de minimis, detrimental effects to the illuminated tissue.

Irrigation of the sphenoidal sinus simultaneously with that of the light treatment and/or during pauses between intermittent light treatments may be required to dissipate heat in order to protect proximate tissues, such as the sphenoidal mucosa from damage.

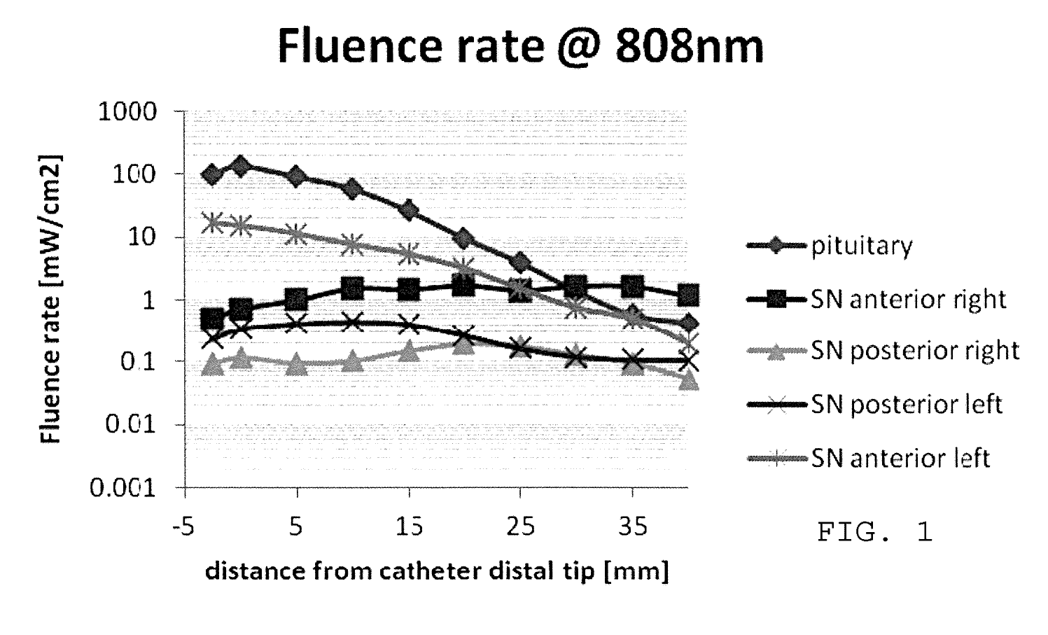

Numerical simulation of light propagation in tissue (similar to the simulation used to create the experimental data shown in FIGS. 1 and 2) may be applied on a patient-by-patient basis, using the individual's anatomic data from MRI, in order to optimize light dosing.

By way of illustrative example, the present invention is shown and described with the optical, system including a light source, an optical fiber, a diffuser and an optical homogenizer. It is, however, contemplated and within the intended scope of the present invention to eliminate the diffuser and optical homogenizer from the optical system. Furthermore, so long as a light source, of sufficient power and wavelength as described herein, is itself able to be advanced so that its distal end is disposed entirely within the sphenoidal sinus then the optical fiber may also be eliminated from the optical system.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication; journal article, book or any other reference cited herein is each incorporated by reference in their entirety.

What is claimed is:

1. A transnasal delivery method for treating or preventing Parkinson's disease using photobiomodulation, comprising the steps of:
providing an optical system including a light source and an optical fiber;
positioning within a nasal cavity a distal end of the optical fiber; and
activating the light source to irradiate substantia nigra brain tissue with an effective amount of light in the treatment or prevention of Parkinson's disease.

2. The method in accordance with claim 1, wherein the light source has a wavelength in a range of approximately 650 nm-approximately 1000 nm.

3. The method in accordance with claim 2, wherein the light source has a wavelength in a range of approximately 670 nm-approximately 810 nm.

4. The method in accordance with claim 1, wherein the positioning step comprises advancing the distal end of the optical fiber so as to be disposed only within a sphenoidal sinus.

5. The method in accordance with claim 1, wherein the positioning step comprises the steps of:
enlarging the sinus; and
transnasal delivery of the optical system to a position in which the distal end of the optical fiber is disposed entirely within the sphenoidal sinus.

6. The method in accordance with claim 1, wherein the substantia nigra is irradiated with a fluence rate between approximately 0.1 mW/cm² and approximately 10 mW/cm² at different locations inside the substantia nigra.

7. The method in accordance with claim 1, wherein a range of approximately 0.1% to approximately 1.0% of irradiated light emitted by the optical system reaches the substantia nigra.

8. The method in accordance with claim 1, wherein the method does not require brain surgery.

9. The method in accordance with claim 8, wherein the method eliminates having an access path through a frontal lobe of the brain.

10. The method in accordance with claim 1, wherein the optical device is not implanted in the brain.

11. The method in accordance with claim 1, wherein a cribriform plate of the brain is not irradiated with light nor does the irradiated light pass across the cribriform plate.

12. The method in accordance with claim 1, wherein the method does not require general anesthesia.

13. The method in accordance with claim 1, wherein the optical system further comprises a diffuser positioned within the nasal cavity.

14. The method in accordance with claim 1, wherein the positioning step comprises advancing the distal end of the optical fiber and the diffuser so that together these components are disposed only within a sphenoidal sinus.

15. The method in accordance with claim 13, wherein the optical system further comprises an optical homogenizer positioned within the nasal cavity.

16. The method in accordance with claim 15, wherein the positioning step comprises advancing the distal end of the optical fiber, the diffuser and the optical homogenizer so that together these components are disposed only within a sphenoidal sinus.

17. The method in accordance with claim 15, wherein the positioning step comprises the steps of:
enlarging the sinus; and
transnasal delivery of the optical system to a position in which the distal end of the optical fiber along with the diffuser and optical homogenizer are disposed entirely within the sphenoidal sinus.

18. A transnasal delivery method for treating or preventing Parkinson's disease using photobiomodulation, comprising the steps of:
providing an optical system including a light source;
positioning within a nasal cavity a distal end of the light source; and
activating the light source to irradiate substantia nigra brain tissue with an effective amount of light in the treatment or prevention of Parkinson's disease.
